(19) United States
(12) Patent Application Publication
(10) Pub. No.: US 2012/0226080 A1
(43) Pub. Date: Sep. 6, 2012

(54) METHOD AND SYSTEM FOR PROVIDING A HYDROCARBON-BASED ENERGY CARRIER USING A PORTION OF RENEWABLY PRODUCED METHANOL AND A PORTION OF METHANOL THAT IS PRODUCED BY MEANS OF DIRECT OXIDATION, PARTIAL OXIDATION, OR REFORMING

(75) Inventor: Roland Meyer-Pittroff, Freising (DE)

(73) Assignee: SILICON FIRE AG, Meggen (CH)

(21) Appl. No.: 13/389,803

(22) PCT Filed: Sep. 9, 2009
(86) PCT No.: PCT/EP2009/061707

§ 371 Date: Apr. 24, 2012

(30) Foreign Application Priority Data
Aug. 13, 2009 (EP) 09167848.2
Aug. 13, 2009 (WO) PCTEP2009060472
Sep. 9, 2009 (WO) PCTEP2009061707

(51) Int. Cl. C07C 29/50; B01J 19/08

(52) U.S. Cl. 568/910.5; 422/162

(57) ABSTRACT

Methanol is produced as a storable and transportable carbon-based energy carrier using at least two different methods. To produce a first methanol portion, water is electrolyzed, preferably using energy from renewable energy sources such as solar or wind power, to produce hydrogen. The hydrogen is then reacted with carbon dioxide to produce the first methanol portion. To produce a second methanol portion, methane is reacted with oxygen. A controller can advantageously be used to control the amount of chemicals in each reaction.

### CROSS-REFERENCE TO RELATED APPLICATIONS

The present application claims the priorities of Patent Cooperation Treaty Application No. PCT/EP2009/061707, as filed on Sep. 9, 2009; and Patent Cooperation Treaty Application No. PCT/EP2009/060472, as filed on Aug. 13, 2009; and European Patent Application No. EP09167848.1, as filed on Aug. 13, 2009, the disclosures of which are incorporated herein by reference in their entirety for all purposes.

### FIELD OF INVENTION

The present application relates to methods and systems for providing storable and transportable carbon-based energy carriers using renewably produced methanol and using methanol that is produced by means of direct oxidation or partial oxidation or reforming.

### BACKGROUND OF THE INVENTION

Carbon dioxide CO2 is a chemical compound made of carbon and oxygen. Carbon dioxide is a colorless and odorless gas. At a low concentration, it is a natural component of the air and arises in living beings during cell respiration, but also during the combustion of carbon-containing substances in the event of sufficient presence of oxygen. Since the beginning of industrialization, the CO2 portion in the atmosphere has risen significantly. The main causes for this are the CO2 emissions caused by humans—so-called anthropogenic CO2 emissions. The carbon dioxide in the atmosphere absorbs a part of the thermal radiation. This property makes carbon dioxide a so-called greenhouse gas (GHG) and one of the contributory causes of the global greenhouse effect.

For these and also other reasons, research and development is currently being performed in greatly varying directions to find a way to reduce the anthropogenic CO2 emissions. In particular in connection with power generation, which is frequently performed by the combustion of fossil energy carriers such as coal, oil, or gas, but also using other combustion processes, for example, waste combustion, there is a great need for reduction of the CO2 emissions. Over 20 billion tons of CO2 per year are discharged into the atmosphere by such processes.

The principle of climate neutrality is sought, in that approaches are being pursued in which attempts are made to compensate for power generation connected with CO₂ emissions through alternative energies. This approach is shown in very schematic form in FIG. 1. Emitters of greenhouse gases (GHG), such as industrial firms (e.g., automobile producers) 1 or power plant operators 2, invest or operate wind farms 3, for example, at other locations in the scope of compensation projects, in order to generate power therein without GHG emissions. Climate neutrality can thus result in absolute terms. Numerous companies are attempting to purchase a “climate-neutral record” in this way

Wind and solar power plants which convert renewable energies into electrical power have an unsteady power delivery, which makes system operation according to the requirements of an integrated electric network extraordinarily difficult and causes system and operating costs for additional reserve and frequency regulation systems. The power generation costs of wind or solar power plants are thus additionally substantially burdened accordingly in relation to power plants which can directly follow the power requirements of the integrated network.

It is considered to be a problem that currently nearly all renewable electrical power which is generated is fed into the public AC voltage integrated network, whose frequency can only vary within very narrow limits (e.g., +/−0.4%). This can only be achieved if the power generation in the network is practically always equal to the consumption. The necessity of always having to maintain the sufficient reserve and frequency regulatory capacities for wind and solar power plants results in a corresponding increase of the cost of the power supply using these systems. Wind and solar power plants in the integrated electric network therefore result in further “hidden” costs and problems.

With the current state of construction of wind power plants in many countries, the electrical power supply network can already be subject to serious problems if, for example, as a result of a lack of wind or strong wind, the wind power fails to a large extent, above all if this failure occurs suddenly and unexpectedly. In any case, however, reserve and frequency regulatory capacities adapted to the installed wind and solar performance are necessary.

Therefore, solar and wind power plants which feed into an integrated electric network can hardly replace the installed performances of other power plants in the integrated network. This has the result that solar and wind power can approximately only be valued at the saved fuel costs of the other thermal power plants present in the network.

According to the priority applications mentioned at the beginning, the renewable forms of energy can be combined particularly advantageously with fossil forms of energy. Such a combination allows hydrocarbon-based energy carriers to be produced in corresponding Silicon Fire™ systems. These Silicon Fire™ systems are particularly capable of producing methanol therewith.

### SUMMARY OF THE INVENTION

The object presents itself of providing a method which is capable of producing storable hydrocarbon-based energy carriers, for example, as fuels or combustibles, in such a way that the (total) CO2 balance of these fuels or combustibles is improved in relation to previously known approaches. The provision of these energy carriers is to be performed with the most minimal possible emission of CO2, and the use of these energy carriers is to contribute to a reduction of the worldwide emission of CO2.

A method and a system (device) for providing storable and transportable energy carriers is provided according to the invention

According to a first embodiment of the invention, carbon dioxide is used as the carbon supplier. The carbon dioxide is preferably withdrawn from a combustion process or an oxidation process of carbon or hydrocarbons by means of CO2 capture. In addition, electrical DC power is provided. The DC power is substantially generated by means of renewable energy, and is used to perform electrolysis of water or an aqueous solution, to thus produce hydrogen and oxygen as intermediate product. The carbon dioxide is then caused to react with the hydrogen, in order to convert these products into a first methanol portion.

A second methanol portion is produced either by direct oxidation (oxidative transformation) of a hydrocarbon, preferably methane-containing natural gas, or from a synthesis gas, which essentially consists of carbon monoxide CO and hydrogen H2. This synthesis gas is produced either by partial oxidation of hydrocarbons, e.g., methane-containing natural gas, with oxygen or by reforming carbon, e.g., coal, or hydrocarbons, e.g., natural gas, oils, or biomasses, with oxygen and/or carbon dioxide and/or water vapor, according to the invention, the oxygen for the direct oxidation or for the production of the synthesis gas essentially or completely originating from the same electrolysis of water or an aqueous solution, using which the hydrogen for the first methanol portion is also produced. This oxygen can be buffered if needed. The reforming can preferably also be performed as autothermal reforming, in which the exothermic partial oxidation and the endothermic reforming using water vapor and/or carbon dioxide are combined so that the combined overall reaction runs without heat exchange with the environment, i.e., autothermally.

Carbon dioxide as the carbon supplier can also be taken according to the invention from crude natural gas, which can have over 10% carbon dioxide portion depending on the natural gas source. Carbon dioxide can also originate from processes of lime burning or calcination to form soda, for example.

According to the invention, the most consistent and long-term possible system operation of a corresponding Silicon Fire™ system is sought, which is achieved by the combination of the production of the first (renewable) methanol portion with the production of the second (substantially fossil) methanol portion.

The Silicon Fire™ system according to the invention is controlled and the individual processes are “linked” with one another so that:

the total yield of the reaction products is as maximal as possible,

and/or the (total) CO2 emission is as minimal as possible,

and/or the most consistent and long-term possible system utilization is achieved,

and/or the product-specific investment and operating costs of the regenerative power plant and the Silicon Fire™ system are as minimal as possible.

According to the invention, the electric power from wind and/or solar power plants is not consumed in an integrated network, but rather converted in a Silicon Fire™ system into methanol as a relatively easily storable and transportable form of energy. i.e., the renewable energies are chemically converted into a relatively easily storable and transportable form of energy.

It is a further advantage of the direct transfer into a relatively easily storable and transportable form of energy that the energy conversion efficiency is increased, since in the case of photovoltaic systems, no inverters are necessary for producing AC voltage and in general a remote transport of the electrical power, which is subject to losses, via long high-voltage lines is not necessary.

In addition, the fossil gaseous raw material methane can be converted in an efficient chemical process directly into liquid methanol, which is again relatively easily storable and transportable in relation to a gas.

The production costs of renewable electrical power from solar and wind power plants are relatively high for the foreseeable future. This has the result that even in the case of direct usage of this electrical power for chemical processes, as proposed here, the chemical products produced solely therewith are substantially more expensive than conventionally—typically fossil—produced products

To remedy this disadvantage, in the Silicon Fire™ system according to the invention, a combination, which is preferably as economically and ecologically optimum as possible, of the production of a first substantially renewable methanol portion with the production of a second preferably substantially fossil methanol portion is implemented, the oxygen required for the production of the second methanol portion substantially or completely originating from the electrolysis process, using which the hydrogen for the first methanol portion is produced.

In times of electrical peak demand in the integrated electric network, the regenerative power can also be fed into the integrated network—to achieve higher profits. This feeding is optional.

Instead of feeding the unsteadily occurring renewable electrical power from wind and/or solar power plants into an integrated electric network and compensating for and regulating its variations and failures through other power plants or storage systems, a chemical Silicon Fire™ system is preferably operated using the electrical power, in order to produce methanol as a relatively easily storable and transportable form of energy.

The production of methanol as a relatively easily storable and transportable form of energy can be reduced or even interrupted at any time. The “chemical system parts” for producing both the first substantially renewable and also the second preferably substantially fossil methanol portion can be reduced or shut down relatively easily and rapidly. The decision-making authority is in the area of responsibility of the operator of the Silicon Fire™ system here.

Methanol can be used as an additional energy buffer. Thus, for example, methanol can be stored to be able to provide additional electrical power in the case of peak power demand in the integrated electric network. Methanol can, as needed, either be combusted in thermal power plants, or electrical power can be generated therewith in fuel cells (e.g., direct methanol fuel cells; referred to as MFC).

If needed, the methanol can be converted catalytically into a cracked gas made of hydrogen and carbon monoxide before the combustion. Advantages result therefrom, which can be inferred from the detailed description.

The present invention is based on hydrogen production with the aid of electrical power, which is generated renewably as much as possible and originates from wind and/or solar power plants, for example, in combination with the reaction of the hydrogen with carbon dioxide to form a first methanol portion. Hydrogen thus does not need to be stored or highly compressed or liquefied by cryogenic cooling and transported over large distances, but rather is used as an intermediate product, which is preferably reacted at the location of its production. According to the invention, an energy-converting process, in which solar energy or wind power is converted into electrical power, follows material-converting (chemical) processes, namely the intermediary provision of hydrogen and the conversion of the hydrogen together with carbon dioxide to form the first methanol portion. The second methanol portion is also produced according to the invention by material-converting (chemical) processes, by direct oxidation of hydrocarbons and/or via partial oxidation of hydrocarbons and/or via reforming of carbon or hydrocarbons, the oxygen required for this purpose substantially or completely originating from the electrolysis for the first methanol portion.

A novel solution in energy technology is provided according to the invention, while complying with corresponding guidelines for energy technology, system technology, and economics, together with the requirement for careful usage of all material, energetic, and economic resources.

### BRIEF DESCRIPTION OF THE DRAWINGS

Further advantageous embodiments can be inferred from the description, the figures, and the dependent claims.

Various aspects of the invention are schematically shown in the drawings, in the figures:

FIG. 1: shows a schematic diagram, which illustrates the principle of climate neutrality through investment in or operation of compensation projects;

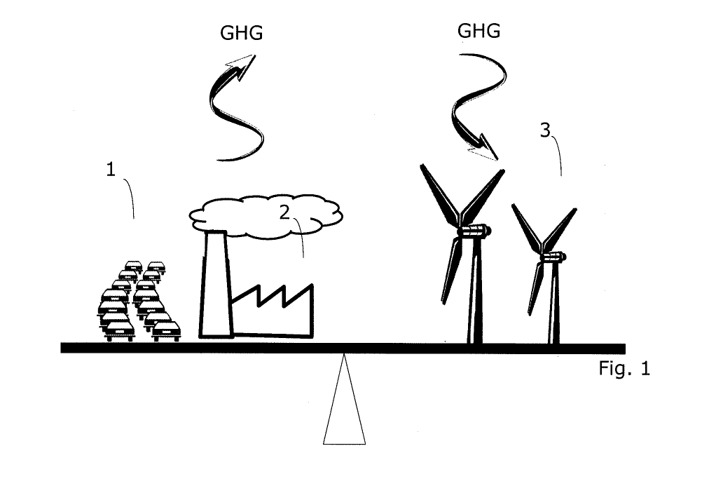

FIG. 2: shows a schematic diagram, which shows the fundamental steps of the method according to the international priority application mentioned at the beginning, or a corresponding Silicon Fire™ system;

FIG. 3: shows a schematic diagram which shows the fundamental steps of the method according to the invention, or a corresponding Silicon Fire™ system.

### DETAILED DESCRIPTION OF THE PREFERRED EMBODIMENTS

The method according to the invention is based on a novel concept, which, employing existing starting materials, provides so-called reaction products, which are either usable directly as energy carriers or are usable indirectly as energy carriers, i.e., after the execution of further steps.

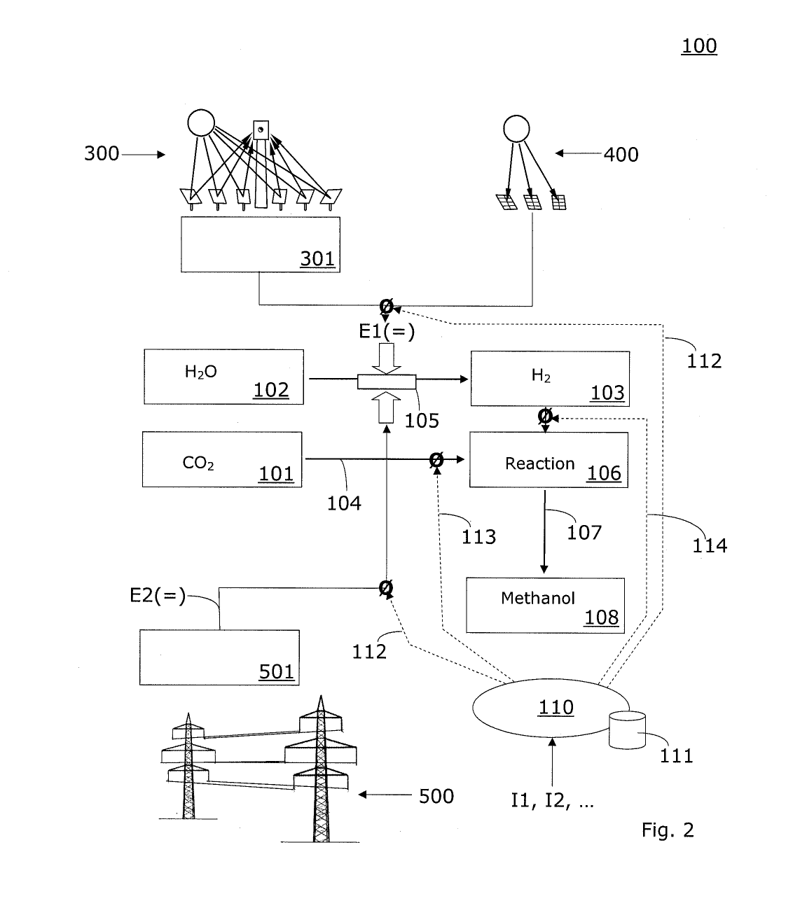

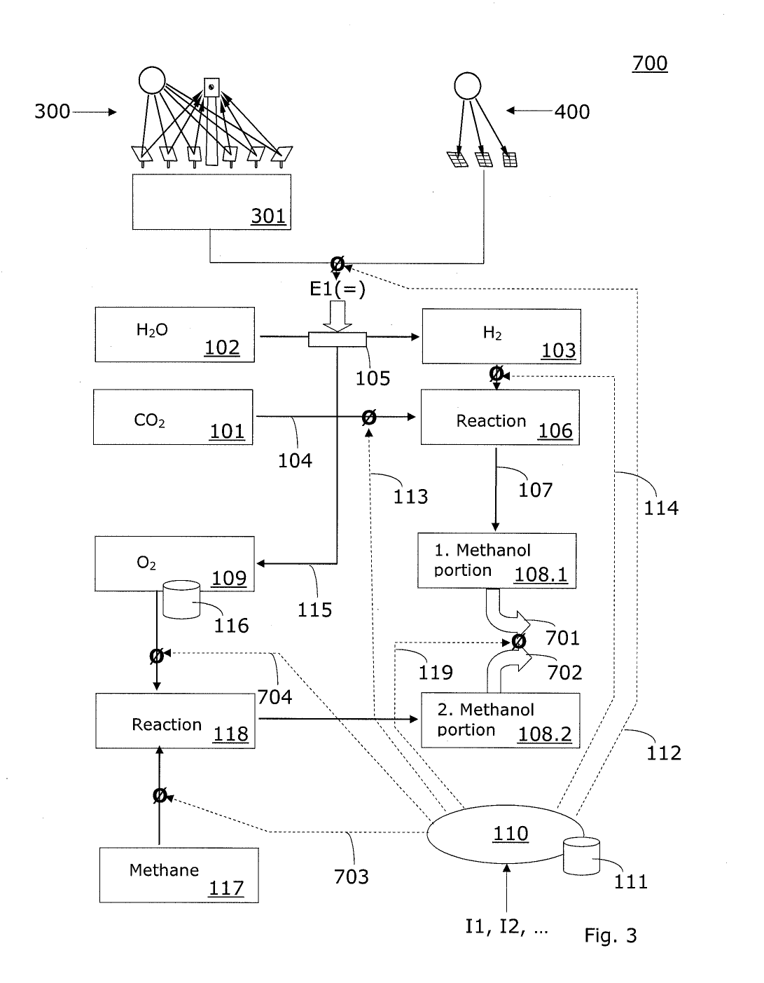

The term energy carrier is used here for materials which can be used directly either as a fuel or combustible. Specifically, as shown in FIGS. 2-3, this relates to methanol 108, 108.1, 108.2.

The transportability of the energy carrier is characterized here by the chemical reaction potential.

In the case of methanol 108.1, 108.2 as the energy carrier, certain boundary conditions are to be maintained during storage and during transport, which are similar to the conditions for handling other fossil liquid fuels and combustibles. The existing infrastructure can be used without problems here. On the material side, certain adaptations may be necessary in order to take the corrosive properties of the methanol into consideration, for example. The safety measures, e.g., with respect to health, fire, and explosion protection, are also to be adapted

FIG. 2 shows a schematic block diagram of the most important modules/components or method steps of a Silicon Fire™ system 100 according to the international priority application mentioned at the beginning This system 100 is designed so that a method for providing storable and transportable energy carriers 108 can be executed. The corresponding method is based on the following fundamental steps.

Carbon dioxide 101 is provided as the carbon supplier. The required electrical DC power E1 is generated here as much as possible by means of renewable energy technology and provided to the Silicon Fire™ system 100. Solar thermal systems 300 and photovoltaic systems 400, which are based on solar modules, are particularly suitable as the renewable energy technology. It is also possible to provide a combination of both system types 300 and 400, since the area required in relation to the electrical power for a solar thermal system 300 is less than that of a photovoltaic system 400.

According to FIG. 2, water electrolysis 105 is performed using the electrical DC power E1, in order to produce hydrogen 103, or hydrogen ions, as an intermediate product.

A system 100 is shown in FIG. 2, which is constructed so that it remedies or compensates for the disadvantages mentioned at the beginning. For this reason, in the Silicon Fire™ system 100 according to the invention, a cost-effective and ecologically optimum combination of renewable power supply (by the systems 300 and/or 400) and conventional power supply, shown by a part of an integrated network 500 here, is preferably implemented. This Silicon Fire system 100 therefore provides using the renewable electrical energy E1 substantially directly in accordance with its occurrence for chemical reactions (the water electrolysis reaction 105 here) and thus chemically bonding and storing it. A further portion of the required energy is obtained from the integrated network 500. This portion is converted into direct current (power) E2. A corresponding converter 501 is used for this purpose, as indicated in schematic form in FIG. 2. The corresponding system parts or components are also designated here as the power supply converter system 501.

The power supply of the system 100 according to FIG. 2 is controlled and regulated by means of an intelligent system controller 110. In principle, the respective instantaneously available excess direct current power portion E2 is drawn from the integrated network 500, while the other power portion (E1 here) is obtained as much as possible from a system including solar power plant 300 and/or 400 (or from a wind power plant). An intelligent reversal of the previous principle thus occurs here, in which the energy variations of renewable energy systems 300, 400 are captured by turning conventional systems on and off. Therefore, additional performance and frequency regulation capacities do not have to be maintained in the integrated network 500 for the renewable power plant systems to operate a Silicon Fire™ system 100. This principle allows the operator of a Silicon Fire™ system 100 to incorporate additional technical and economic parameters in the control of the system 100. These parameters are so-called input variables I1, I2, etc., which are incorporated by the controller 110 in decisions. A part of the parameters can be predefined within the controller 110 in a parameter memory 111. Another part of the parameters can come from the outside. This can include, for example, price and/or availability information from the operator of the integrated network 500.

A system 700 according to the invention is shown in FIG. 3, which is constructed so that the disadvantages mentioned at the beginning are remedied or compensated for. A part of this system 700 corresponds to the system 100 according to FIG. 2. Therefore, reference is made in this regard to the preceding description of the corresponding elements.

As described, hydrogen 103, which is reacted to form a first methanol portion 108.1, is produced by water electrolysis 105. The energy for this purpose originates entirely or substantially (preferably more than 80%) from renewable power sources 300 and/or 400. During the water electrolysis 105, oxygen 109 arises as a “byproduct”. This oxygen 109, or a correspondingly gas containing a lot of oxygen, can be withdrawn from the electrolysis system 105 via an oxygen withdrawal 115. An optional oxygen buffer 116 is shown in FIG. 3. Methane or methane-containing gas 117 is provided in the embodiment shown. Another hydrocarbon-containing gas can also be used here. This methane or methane-containing gas 117 can originate from a gas delivery system or, for example, from a biogas system. Methane, or the methane-containing gas 117, is now reacted in a reaction system 118 (e.g., by means of direct oxidation) to form a second methanol portion 108.2. The first methanol portion 108.1 and the second methanol portion 108.2 can be combined (mixed). This optional step is shown in FIG. 3 by the reference signs 701 and 702. The controller 110 can regulate this procedure and adjust the dosage of the portions 108.1 and 108.2, as shown by the optional control or signal line 119.

An array of further control or signal lines can be provided, as shown on the basis of the lines 112, 113, and 114 shown as examples. These lines 112, 113, and 114 control energy or mass streams. In order to be able to regulate the mass streams on the input side of the reaction system 118, optional control or signal lines 703 and 704 can be provided.

So-called software-based decision processes are implemented in the system controller 110. A processor of the controller 110 executes control software and makes decisions in consideration of parameters. These decisions are converted into switching or control commands, which cause the control/regulation of energy and mass streams via control or signal lines 112, 113, 114, 119, 703, 704, for example.

According to one embodiment of the invention, carbon dioxide 101 is used as the carbon supplier, as schematically indicated in FIG. 3. The carbon dioxide 101 is preferably withdrawn from a combustion process or an oxidation process via CO2 capture (e.g., a Silicon Fire™ flue gas purification system). The carbon dioxide 101 can also be separated from crude natural gas and provided, however. Furthermore, electrical DC power E1 is provided. The DC power E1 is generated substantially renewably (e.g., by one of the systems 300 and/or 400 in FIG. 3). The DC power E1 is used to perform water electrolysis, to produce hydrogen 103 as an intermediate product. The electrolysis system, or the performance of such an electrolysis, is identified in FIG. 3 by the reference sign 105. The carbon dioxide 101 is then caused to react with the hydrogen 103 (e.g., by methanol synthesis), in order to react the (intermediate) products 101, 103 to form methanol 108.1. The reaction can be performed in a reaction container 106, and the withdrawal or the provision of the methanol is identified in FIG. 3 by the reference sign 107.

Further fundamental details of this method and the corresponding Silicon Fire™ system 700 are described hereafter.

Water electrolysis employing direct current E1 is suitable in order to be able to produce hydrogen 103 as an intermediate product. The required hydrogen 103 is produced in an electrolysis system 105 by the electrolysis of water H2O 102:

H2O−286.02 kJ=H2+0.5O2. (Reaction 1)

The required (electrical) power E1 for this reaction of 286.02 kJ/mol corresponds to 143,010 kJ per kilogram H2.

The synthesis of the methanol 108.1 (CH3OH) runs in the Silicon Fire™ system 700 according to the exothermic reaction between carbon dioxide 101 (CO2) and hydrogen 103 (H2), as follows:

CO2+3H2=CH3OH+H2O−49.6 kJ (gaseous methanol). (Reaction 2)

The corresponding reaction heat W1 of 49.6 kJ/mol=1550 kJ per kilogram methanol=0.43 kWh per kilogram methanol is dissipated from the corresponding synthesis reactor 106. Typical synthesis conditions in the synthesis reactor 106 are approximately 50 bar and approximately 270° C., so that the reaction heat W1 can also be used, for example, for a seawater desalination system or heating system located nearby.

The methanol synthesis 106 is preferably performed using catalysts, in order to keep reaction temperature and pressure and reaction time low and in order to ensure that high-quality (pure) methanol 108.1 results as the reaction product.

In another preferred embodiment of the invention, methanol synthesis is performed according to an electrolysis method propagated by Prof. George A. Olah. Details in this regard can be taken, for example, from the book “Beyond Oil and Gas: The Methanol Economy”, George A. Olah et al., Wiley-VCH, 1998, ISBN 0-471-14877-6, chapter 11, page 196. Further details can also be inferred from US Patent Application US 2009/001-4336 A1. Prof. George A. Olah describes the methanol synthesis by electrolysis of CO2 and H2O as follows:

CO2+2H2O−682.01 kJ=CH3OH+1.5O2. (Reaction 3)

In this reaction, CO and H2 arise in an intermediate step in a ratio of approximately 1:2. In addition, O2 109 arises, which is used further in the reaction system 118. The CO and H2 which arises on a cathode can be reacted using a copper-based or nickel-based catalyst to form methanol. The synthetic pathway according to reaction 3 is connected with a theoretical supply of 682.01 kJ=0.189 kWh electrical power per mol of produced methanol 108.1.

If the Silicon Fire™ system 700 is located close to a CO2 source, liquefaction of CO2 for the transport can be dispensed with. Otherwise, it is relatively simple according to the prior art to liquefy the CO2 and also to move it over large distances to a Silicon Fire™ system 700. If liquefaction is omitted, and optionally storage and transport over a long distance, the CO2 is conceivably available cost-neutral in consideration of CO2 avoidance credits. The costs for the “acquisition” of the CO2 are also relatively low in case of a transport.

If the reaction system 118 of the Silicon Fire™ system 700 is located close to the O2 source (e.g., the electrolyzer 105 here), liquefaction for transport and long-range transport of even O2 109 can be omitted.

It is indicated in FIG. 3 on the basis of the dashed arrow 112, which originates from the controller 110, that the controller 110 regulates the energy stream E1. The arrow 112 represents a control or signal line. Other possible control or signal lines 113, 114, 703, 704 are also shown. For example, the control or signal line 113 regulates the CO2 quantity which is available for the reaction 106. For example, if less hydrogen 103 is produced, less CO2 must also be supplied. The optional control or signal line 114 can regulate the H2 quantity, for example. Such a regulation is advisable, for example, if there is a hydrogen buffer, from which hydrogen 103 can be withdrawn, even if no hydrogen or less hydrogen is being produced at the moment by electrolysis 105. The optional control or signal line 703 can regulate the supply of the methane 117 and the optional control or signal line 704 can regulate the supply of the oxygen 109.

Experiments have shown that it is particularly cost-effective and environmentally advisable if the Silicon Fire™ system 100 is designed or controlled so that between 15% and 40% is produced as the methanol portion 108.1 from renewable power, while the remaining 85% to 60% is provided as the methanol portion 108.2 from hydrocarbon sources (e.g., from methane 117).

An embodiment of the system 100 is particularly preferred which provides the acquisition of cost-effective electrical power in the low load times from an integrated network 500 (as in FIG. 2).

The aspects of the various embodiments may be combined without problems by a corresponding design of the controller 110.

Further fundamental aspects of the method according to the invention for providing storable and transportable energy carriers are shown hereafter.

The synthesis of regenerative methanol CH3OH from carbon dioxide CO2 and hydrogen H2 is known, which is produced via the (endothermic) electrolysis of water using renewable electrical power according to reaction 1, as already mentioned above.

H2O−286.02 kJ=H2+0.5O2. (Reaction 1)

The exothermic methanol synthesis (reaction 2, as already mentioned above) is represented by the summation formula:

CO2+3H2=CH3OH+H2O−49.6 kJ (gaseous methanol). (Reaction 2)

The renewable electrical power E1 used has the disadvantages in particular in the case of wind and solar power that it is relatively costly and is only available irregularly and with time restrictions

Therefore, it is proposed here that the renewable production of methanol using renewable electrical power E1 according to reactions 1 and 2 be supplemented by a methanol synthesis (in the reaction system 118) based on the raw material natural gas having the main component methane CH4 117, or based on other carbon starting materials or hydrocarbon starting materials. This synthesis can be performed, for example, by exothermic direct oxidation (oxidative transformation, oxidative conversion) of the methane 117 according to the summation formula:

CH4+0.5O2=CH3OH−167.5 kJ/mol. (Reaction 4)

This reaction 4 is referred to here as direct oxidation.

However, the pathway via partial oxidation (see reaction 5) or reforming (see reactions 7 and/or 8) (preferably as autothermal reforming) can also be selected, in order to arrive at the second methanol portion 108.2 via the pathway of synthesis gas (essentially comprising carbon monoxide and hydrogen here).

The fact that the (pure) oxygen 109 or the gas containing a lot of oxygen, for reaction 4 or also for the partial oxidation or the reforming, originates directly or in buffered form from the hydrolysis reaction 1 or the reaction 3 is novel above all here. If the (pure) oxygen 109, or the gas containing a lot of oxygen, is taken from the hydrolysis reaction 1, according to the mass ratios of the reactions 1, 2, and 4, the oxygen from reaction 1 is sufficient to provide at most three times more methanol 108.2 using reaction 4 than using reaction 2, or, if all oxygen 109 from reaction 1 is used, all of the produced methanol originates one-fourth from the “renewable” reaction and three-fourths from the “fossil” reaction 4 (e.g., the direct oxidation).

By reducing the methanol portion from reaction 4, the “renewable” portion 108.1 of the total methanol production can be increased, e.g., also having corresponding effects on the CO2 balance of the overall methanol production and the specific CO2 emissions during the combustion of the “total” methanol for heat generation or as a fuel.

In practice, the available renewable electrical power E1 is preferably maximally utilized to produce “renewable” methanol 108.1 according to reactions 1 and 2 and the portion of the “fossil” methanol 108.2 produced according to reaction 4 is adjusted according to economic and ecological targets and boundary conditions up to the maximum possible value, e.g., according to the desired specific CO2 emission of the “total” methanol in the case of combustion or according to the current price and the availability of the natural gas or according to the “total” quantity of methanol to be produced or according to the prices of the various methanol portions 108.1, 108.2.

It is particularly advantageous that the “renewable” methanol 108.1 and the “fossil” methanol 108.2 can occur separately in various reactors 106, 118 and can either be discharged separately or can be mixed after the occurrence and optional temporary storage in arbitrary portions (see arrows 701, 702), so that the Silicon Fire™ system 700 can supply pure “renewable” methanol and pure “fossil” methanol, but also arbitrary mixtures of both, in order to be able to be marketed, e.g., as renewable fuel having permissible fossil portion or permissible specific CO2 emission.

The production ratio of one-fourth “renewable” methanol 108.1 and three-fourths “fossil” methanol 108.2 corresponds to the computed solar (peak) energy yield in relation to the duration of a calendar year, i.e., the Silicon Fire™ system 700 could mathematically produce “renewable” methanol during approximately one-fourth of the duration of a year and “fossil” methanol during approximately three-fourths of the duration of a year, if the oxygen 109 required for the “fossil” methanol is buffered accordingly. The buffer 116 can be used for this purpose.

The reaction heat of the exothermic reactions 2 and 4 is preferably used, whereby the specific CO2 emissions to be credited of the various methanol fractions 108.1, 108.2 would also be reduced. For example, under the conditions of the Persian Gulf, heating of thermal seawater desalination systems comes very advantageously into consideration for the heat usage, in particular during the colder time of year, if, because of the substantially lower power demand for building air conditioning, thermal power plants are run at part load or even shut down and their waste heat is no longer (sufficiently) available for the seawater desalination systems connected thereto.

The use of the large oxygen mass stream arising during the electrolysis 105 for reaction 4 or for the partial oxidation or the reforming avoids the ecological risks which can otherwise occur in the event of emission into the environment.

Reaction 4 is mentioned, e.g., in Olah, G. et al., “Behind Oil and Gas: The Methanol Economy”, Wiley-VCH Verlag, 2006, pages 171 and 172 as “direct oxidative transformation of natural gas to methanol” or as “producing methanol from natural gas.”

A two-stage oxidation process which runs at 500 and 350° C. is described by Mantashyan, A. A. et al. in “Non isothermal oxidative conversion of methane to methanol . . . ”, Hayastani Kimiakan Handes (2007), 60(5), pages 887-897. Further recent literature sources for reaction 4 are, e.g.:

Holmen, A., “Direct conversation of methane to fuels and chemicals”, Catalysis Today (2009), 142(1-2), page 8.

Ankan, P. et al., “Mechanism and energetics of methane to methanol conversation”, Organometallics (2007), 26(4), 793-809.

However, the result of reaction 4 can also be achieved by the combination of the following industrially tested reactions 5 and 6, for example:

The partial oxidation of methane 117:

CH4+0.5O2=CO+2H2−36 kJ/mol. (Reaction 5)

and classical methanol synthesis (e.g., 250 bar, 350° C., using chromium oxide-zinc oxide catalyst):

CO+2H2=CH3OH−128.20 kJ/mol (liquid methanol). (Reaction 6).

In the event of the combination of reactions 5 and 6, a quasi-oxidation of the methane-containing gas 117 also occurs using oxygen-containing gas 109.

Under certain circumstances, reaction 6 can run in the same reactor as reaction 2, possibly with change or adaptation of the catalyst filler and the catalysis conditions.

The reforming of methane using water steam (steam reforming) to form synthesis gas runs in an endothermic manner according to the following reaction:

CH4+H2O (gaseous)=CO+3H2+206.2 kJ/mol. (Reaction 7)

The reforming of methane using carbon dioxide to form synthesis gas runs in an endothermic manner according to the following reaction:

CH4+CO2=2CO+2H2+247 kJ/mol. (Reaction 8)

The three reactions 5, 7, and 8 can run jointly in a reactor at temperatures of approximately 800-1000° C. via catalysts and may be controlled so that they run with energy autonomy as much as possible (“autothermally”) and the reaction products result in a suitable synthesis gas for the classical methanol synthesis according to reaction 6.

Further details can be inferred, for example, from the publication of Bharadwaj, S. S.; L. D. Schmidt: Catalytic partial oxidation of natural gas to syngas. Fuel Processing Technology 42 (1995), pages 109-127.

According to the invention, CO2 101 is used as the starting material and carbon supplier for the methanol synthesis in the reactor 106. Steam reforming systems, CO2 capture systems for crude natural gas, lime burning furnaces, calcination systems for soda, fermentation systems for bioethanol, seawater desalination systems, large boiler systems for fossil fuels (e.g., power plant boilers), and other systems or combustion processes which emit relatively large quantities of CO2 are preferably used as the CO2 sources.

The invention allows the substantial economic disadvantages of known approaches to be avoided if—as in the case of the Silicon Fire™ system 700—the unsteadily yielded electrical solar and/or wind energy is converted directly into chemical reaction enthalpy and is stored chemically bound, without additional capacities for reserve performances and/or frequency regulation in the integrated network and the expenditures required for this purpose being necessary.

In the case of photovoltaic power generation by means of a photovoltaic system 400, it is a further advantage that the direct current E1 primarily yielded from the solar cells of the photovoltaic system 400 can be used directly for the chemical process (electrolysis 105), without having to be converted into alternating current for voltage transformation via converters.

In another embodiment of the invention, an ATR process (autothermal reforming) can also be used for the processing of a starting material. In the case of autothermal reforming, a hydrocarbon-containing or a carbon-containing starting material is oxidized in a reaction zone in the presence of a substoichiometric quantity of oxygen (e.g., oxygen 109), which is not sufficient for complete oxidation. In addition, water steam and/or carbon dioxide are supplied, in order to be able to produce synthesis gas in this way, which essentially comprises carbon monoxide and hydrogen. The starting material can be natural gas or another hydrocarbon. It is also possible to convert coals, oils, combustion gases, biomasses, oil sands, or oil shales using the ATR process to form a suitable synthesis gas.

In another embodiment of the invention, carbon dioxide is supplied during the ATR process. The addition of CO2 can be advantageous if the stoichiometric ratios are not optimal for the synthesis of methanol because of the starting materials.

In the embodiments which are based on an ATR process, synthesis gas is produced, which essentially comprises carbon monoxide and hydrogen. Methanol is then synthesized from the synthesis gas in a downstream step, as shown in reaction 6, for example.

In a particularly preferred embodiment of the invention, the methanol 108.1, 108.2 is used as an energy carrier for storage and transport.

Carbon dioxide as a carbon supplier can also be withdrawn according to the invention from the crude natural gas, which can have greater than 10% carbon dioxide portion depending on the natural gas source. After the conveyance of the crude natural gas, gas washing (by means of gas washing technology or another gas separation technology) is already currently typically performed, in order to separate the CO2 from the actual natural gas. This CO2 is typically emitted into the atmosphere. According to the invention, the CO2, which is provided in substantially pure form, can be used as a carbon supplier 101.

Catalytic cleavage (e.g., at approximately 380° C.) before the combustion according to reaction 9:

CH3OH (liquid)=2H2+CO+128.20 kJ/mol. (Reaction 9)

can provide the following advantages for the usage of the methanol 108.1, 108.2 in internal combustion engines, e.g., also in combined gas/steam turbine systems.

An internal combustion engine normally operated using gas does not need to be converted for a liquid fuel. The reaction heat of the endothermic methanol cleavage reaction 9 of 128.20 kJ/mol or 4006 kJ/kg methanol can be applied by the exhaust gas waste heat of an internal combustion engine, whereby the original heating value of the methanol of 19,900 kJ/kg increases by the supplied reaction heat by approximately 20%. The application of a corresponding methanol cleavage for power plant processes is described, for example, in Muβenbrock, K: “Möglichkeiten zur Nutzung von Methanol in Kraftwerksprozessen [Possibilities for Using Methanol in Power Plant Processes]”, VGB Kraftwerkstechnik 71 (1991), issue 8, page 759-764.

List of Reference Numerals:
1 vehicle industry/automobile construction
2 power plant operator
3 wind farm
100 Silicon Fire™ system (from parallel application)
101 carbon dioxide
102 water
103 hydrogen
104 provision of carbon dioxide
105 performance of electrolysis
106 combination of the hydrogen (H₂) and the carbon dioxide/synthesis reactor
107 discharge/provision of methanol
108 transportable methanol energy carrier
109 oxygen or oxygen-containing gas
110 (system) controller
111 parameter memory
112, 113, 114 control or signal lines
115 oxygen withdrawal
116 optional oxygen buffer
117 methane or methane-containing gas
118 reaction system
119 control or signal line
300 solar thermal system
301 conversion of heat into direct current
400 solar plant (photovoltaic system)
500 integrated network
501 conversion of AC voltage into direct current (power supply system)
700 Silicon Fire™ system (invention)
701, 702 methanol flows
703, 704 control or signal lines
E1 DC power
I1, I2, etc. input variables

What is claimed is:

1. A method for providing storable and transportable energy carriers (108) comprising the following steps:
providing carbon dioxide (CO2) as a carbon supplier,
providing electrical DC power, which is generated by means of renewable energy technology,
performing a water electrolysis using said electrical DC power to produce hydrogen as an intermediate product,
combining the hydrogen (H2) and the carbon dioxide (CO2), to react them to form a first methanol portion,
providing gas containing oxygen, which arises during the performance of said water electrolysis,
providing hydrocarbon-containing gas,
performing an oxidation of the hydrocarbon-containing gas using the gas containing oxygen to form a second methanol portion.

2. The method according to claim 1, wherein, the first methanol portion is combined with the second methanol portion to obtain said storable and transportable energy carrier.

3. The method according to claim 1, wherein said hydrocarbon containing gas originates from a biogas system.

4. The method according to claim 1, wherein the gas containing oxygen is temporarily stored in a buffer.

5. The method according to claim 1 wherein carbon dioxide (CO2) is provided from a combustion process or an oxidation process.

6. The method according to claim 1 wherein carbon dioxide (CO2) is provided from a system which frees crude natural gas from a carbon dioxide portion during the purification.

7. The method according to claim 1 wherein a portion of said electrical DC power is generated by a wind power plant.

8. The method according to claim 1 wherein a portion of said electrical DC power is delivered by means of a solar thermal system and/or a photovoltaic system.

9. The method according to claim 1 wherein said hydrocarbon-containing gas is a methane-containing gas.

10. The method according to claim 1 wherein said hydrocarbon-containing gas is produced from carbon-containing material.

11. The method according to claim 1 wherein said second methanol portion is produced by means of reforming via the pathway of synthesis gas.

12. The method according to claim 11, wherein said reforming is autothermal reforming.

13. The method according to claim 1 wherein the first methanol portion and/or the second methanol portion are reacted to form carbon monoxide and hydrogen.

14. The method according to claim 1 wherein before the reaction to form the second methanol portion, a synthesis gas is produced by a partial oxidation of hydrocarbons, which consists essentially of carbon monoxide CO and hydrogen H2

15. A system for providing a storable and transportable energy carrier comprising:
a system for generating a first power portion in the form of DC power from at least one renewable power source,
an electrolysis device for providing hydrogen as an intermediate product and for providing gas containing oxygen the energy demand of this electrolysis device coming from said first power portion,
a carbon dioxide supply,
a first reactor for producing a first methanol portion by reacting hydrogen with said carbon dioxide,
a second reactor for producing a second methanol portion by reacting oxygen with a hydrocarbon-containing gas.

16. The system according to claim 15 wherein-the first methanol portion is mixed with the second methanol portion.

17. The system according to claim 15 further comprising a controller to mix the chemicals in proper proportions.

18. The system according to claim 17 wherein a software-based decision process is implemented in the controller so that the first methanol portion is produced in greater quantity than the second methanol portion.

19. The system according to claim 18 wherein a software-based decision process is implemented in the controller, so that approximately one-fourth of the combined methanol portion is the first methanol portion.

20. The system according to claims 17 wherein energy and/or mass streams are regulated using the controller via signal lines.

21. The system according to claims 15 wherein a synthesis gas including carbon monoxide CO and hydrogen H2 is produced in said second reactor prior to said second methanol portion.

22. The system according to claim 15 wherein reforming reactions are used in said second reactor to form said second methanol portion.

23. The system according to claim 22 wherein the reforming is autothermal reforming.

24. The system according to claim 15 wherein cracked gas including carbon monoxide and hydrogen is produced from the first methanol portion and/or the second methanol portion.
